(19) United States
(12) Patent Application Publication
(10) Pub. No.: US 2024/0101523 A1
(43) Pub. Date: Mar. 28, 2024

(54) NOVEL BENZOXAZINE COMPOUND, RESIN RAW MATERIAL COMPOSITION CONTAINING THE SAME, CURABLE RESIN COMPOSITION, AND CURED PRODUCT THEREOF

(71) Applicant: HONSHU CHEMICAL INDUSTRY CO., LTD., Chuo-ku, Tokyo (JP)

(72) Inventor: Yoshimi UTAKA, Wakayama-shi (JP)

(21) Appl. No.: 18/272,055

(22) PCT Filed: Jan. 24, 2022
(86) PCT No.: PCT/JP2022/002311

§ 371 Date: Jul. 12, 2023

(30) Foreign Application Priority Data
Jan. 29, 2021 (JP) 2021-013396

(51) Int. Cl. C07D 265/16; C07D 413/06; C08G 73/02; C08L 63/00

(52) U.S. Cl. CPC C07D 265/16; C07D 413/06; C08G 73/0233; C08L 63/00; C08L 2201/08

(57) ABSTRACT

An object is to provide a novel benzoxazine compound that has high heat resistance and can cure under low temperature conditions, a resin raw material composition containing the benzoxazine compound, a curable resin composition, and a cured product thereof. As a solution, a benzoxazine compound represented by general formula (1) is provided:

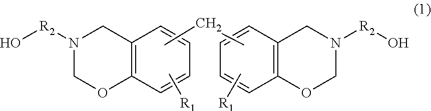

wherein R₁ represents a hydrogen atom or an alkyl group having 1 to 6 carbon atoms, and R₂ represents an alkylene group having 1 to 6 carbon atoms.

### TECHNICAL FIELD

The present invention relates to a novel benzoxazine compound, a resin raw material composition containing the benzoxazine compound, a curable resin composition, and a cured product thereof. The invention particularly relates to a novel benzoxazine compound having benzoxazine rings at both ends of a methylene group and further having a hydroxy group, a resin raw material composition containing the benzoxazine compound, a curable resin composition, and a cured product thereof.

### BACKGROUND ART

Benzoxazine compounds, which are compounds synthesized by reacting a phenol, an amine, and formaldehyde, are known as thermosetting resin raw materials that, when heated, undergo ring-opening polymerization of a benzoxazine ring to cure without producing any volatile by-products, and are used as raw materials of a molded body usable as a material for an insulating substrate, a liquid crystal alignment agent, a resin composition for semiconductor sealing, and the like. In these applications, heat resistance excellent in stability and reliability at high temperatures is required.

On the other hand, benzoxazine compounds typically have relatively high curing temperatures, and to achieve lower polymerization temperatures, catalysts, polymerization accelerators, and in addition highly reactive benzoxazine compounds have recently been developed. Among the highly reactive benzoxazine compounds, a benzoxazine composition that can be cured in an environmentally friendly process at relatively low temperatures in short time periods and contains a hydroxyl functional group or a nitrogen-containing heterocycle has been reported (PTL 1).

### CITATION LIST

### Patent Literature

PTL 1: Japanese Unexamined Patent Application Publication (Translation of PCT Application) No. 2011-530570

### SUMMARY OF INVENTION

### Technical Problem

To lower the temperature during the process of molding a thermosetting resin, thereby achieving higher efficiency due to reduced time of heating and cooling and saved energy and suppressing material thermal degradation due to exposure to high temperature during polymerization, excellent materials that can cure under lower temperature conditions are demanded.

It is an object of the present invention to provide a novel benzoxazine compound that has high heat resistance and can cure under low temperature conditions, a resin raw material composition containing the benzoxazine compound, a curable resin composition, and a cured product thereof.

### Solution to Problem

To achieve the above object, the present inventors have conducted intensive studies and found that a novel benzoxazine compound obtained by using bisphenol F as a raw material, having benzoxazine rings at both ends of a methylene group, and further having a hydroxy group has high heat resistance and can cure under low temperature conditions, thereby completing the present invention.

The present invention is as follows.
1. A benzoxazine compound represented by general formula (1) below.

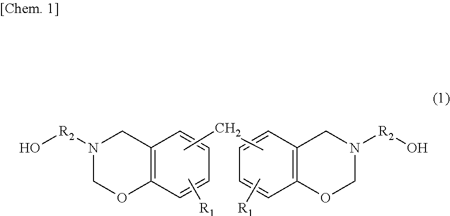

(In the formula, R₁ represents a hydrogen atom or an alkyl group having 1 to 6 carbon atoms, and R₂ represents an alkylene group having 1 to 6 carbon atoms.)
2. A resin raw material composition containing the benzoxazine compound according to 1.
3. A curable resin composition containing the benzoxazine compound according to 1. or the resin raw material composition according to 2.
4. The curable resin composition according to 3., containing the benzoxazine compound according to 1. or the resin raw material composition according to 2., and at least one selected from the group consisting of epoxy resins, benzoxazine compounds other than the benzoxazine compound represented by general formula (1), phenol resins, and bismaleimide compounds.
5. A cured product obtained by curing the curable resin composition according to 3. or 4.

### Advantageous Effects of Invention

The inventive compound, because of being able to cure at a lower temperature than Comparative Example Compound A having the following chemical structure known in the art, can lower the temperature during the process of molding a thermosetting resin, enabling higher efficiency due to reduced time of heating and cooling and saved energy, and in addition can be used also for a heat-sensitive material (substrate), and thus is very useful.

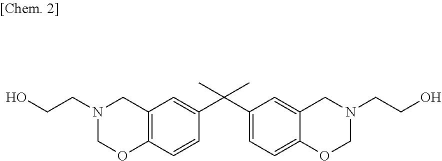 [SMILES: CC(C)(c1ccc2c(c1)CN(CCO)CO2)c1ccc2c(c1)CN(CCO)CO2]

The cured product of the inventive compound has much higher heat resistance than a cured product of Comparative Example Compound A above known in the art, and thus is a material excellent in stability and reliability at high temperatures and is very useful.

The novel benzoxazine compound, the resin raw material composition containing the benzoxazine compound, the curable resin composition, and the cured product thereof in the present invention are suitable for use as resin raw materials for varnishes that can be applied to various substrates, prepregs impregnated with varnishes, print circuit boards, sealants for electronic components, electrical and electronic molded parts, automotive parts, laminated materials, paints, resist inks, and the like.

### DESCRIPTION OF EMBODIMENTS

### Novel Benzoxazine Compound According to Present Invention

A novel benzoxazine compound according to the present invention is represented by general formula (1) below.

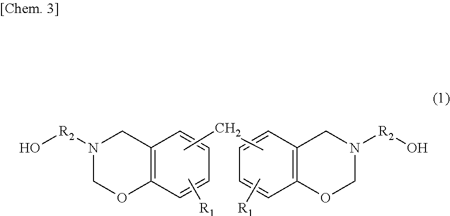

(In the formula, R₁ represents a hydrogen atom or an alkyl group having 1 to 6 carbon atoms, and R₂ represents an alkylene group having 1 to 6 carbon atoms.)

R₁ in general formula (1) is preferably a hydrogen atom or an alkyl group having 1 to 4 carbon atoms, more preferably a hydrogen atom or an alkyl group having 1 carbon atom (methyl group), particularly preferably a hydrogen atom. The structure in the case where R₁ is a hydrogen atom is represented by general formula (1′) below.

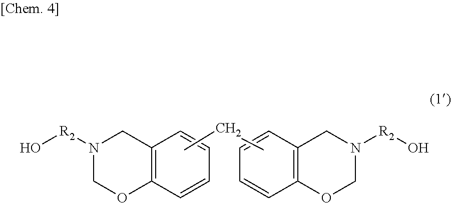

(In the formula, R₂ is as defined in general formula (1).)

When R₁ bonded to each benzene ring in general formula (1) is an alkyl group (R₁′), the bonding position thereof is preferably the ortho position to the bonding position of each oxygen atom. The structure in this case is represented by general formula (1″) below.

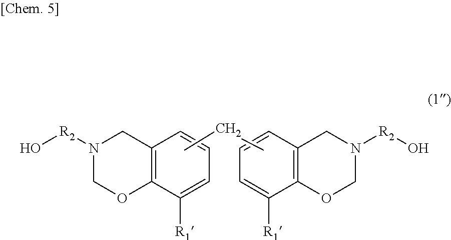

(In the formula, R₁′ represents an alkyl group, and R₂ is as defined in general formula (1).)

R₂ in general formula (1) is preferably an alkylene group having 1 to 4 carbon atoms, more preferably an alkylene group having 1 or 2 carbon atoms, particularly preferably an alkylene group having 2 carbon atoms (ethylene group).

The position of bonding of the methylene group at the center in general formula (1) to the two benzoxazine rings is preferably the ortho or para position to the bonding position of each oxygen atom.

As specific examples of the novel benzoxazine compound represented by general formula (1) in the present invention, compounds (p-1) to (p-32) having the following chemical structures are shown. Of these, compounds (p-1) to (p-20) are preferred, compounds (p-1) to (p-16) are more preferred, compounds (p-1) to (p-12) are still more preferred, and compounds (p-4) to (p-6) are particularly preferred.

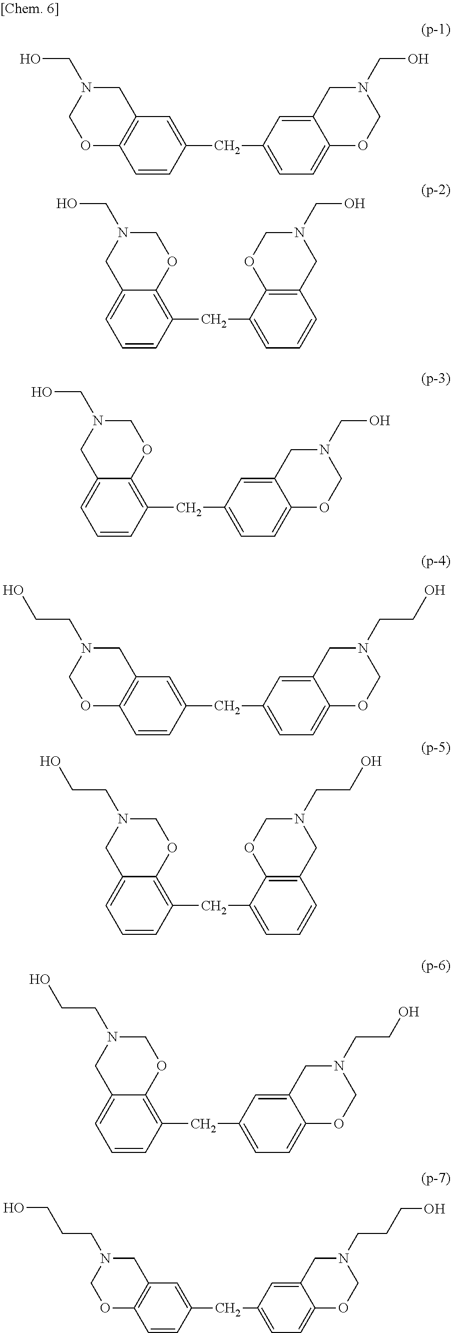

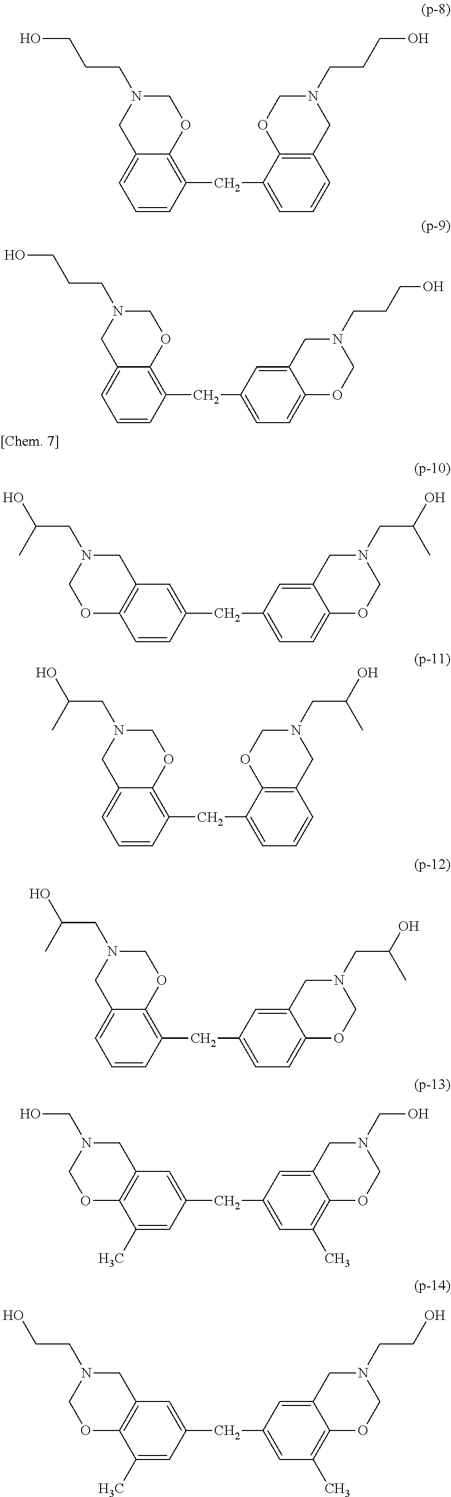

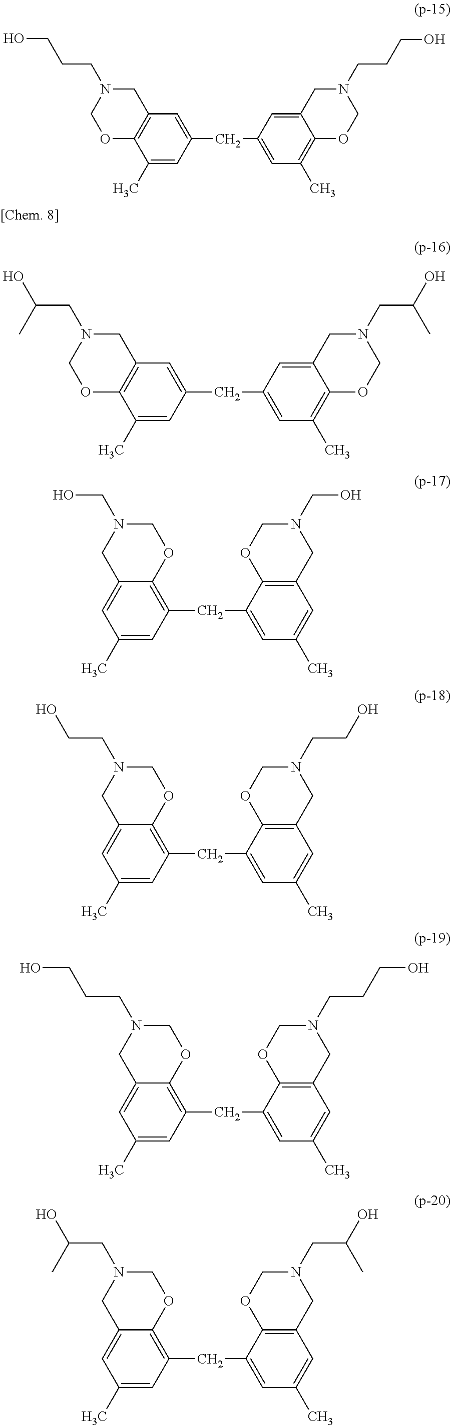 [SMILES: Cc1cc(Cc2cc(C)c3c(c2)CN(CCCO)CO3)cc2c1OCN(CCCO)C2 ; Cc1cc(Cc2cc(C)c3c(c2)CN(CC(C)O)CO3)cc2c1OCN(CC(C)O)C2 ; Cc1cc(Cc2cc(C)cc3c2OCN(CO)C3)c2c(c1)CN(CO)CO2 ; Cc1cc(Cc2cc(C)cc3c2OCN(CCO)C3)c2c(c1)CN(CCO)CO2 ; Cc1cc(Cc2cc(C)cc3c2OCN(CCCO)C3)c2c(c1)CN(CCCO)CO2 ; Cc1cc(Cc2cc(C)cc3c2OCN(CC(C)O)C3)c2c(c1)CN(CC(C)O)CO2]

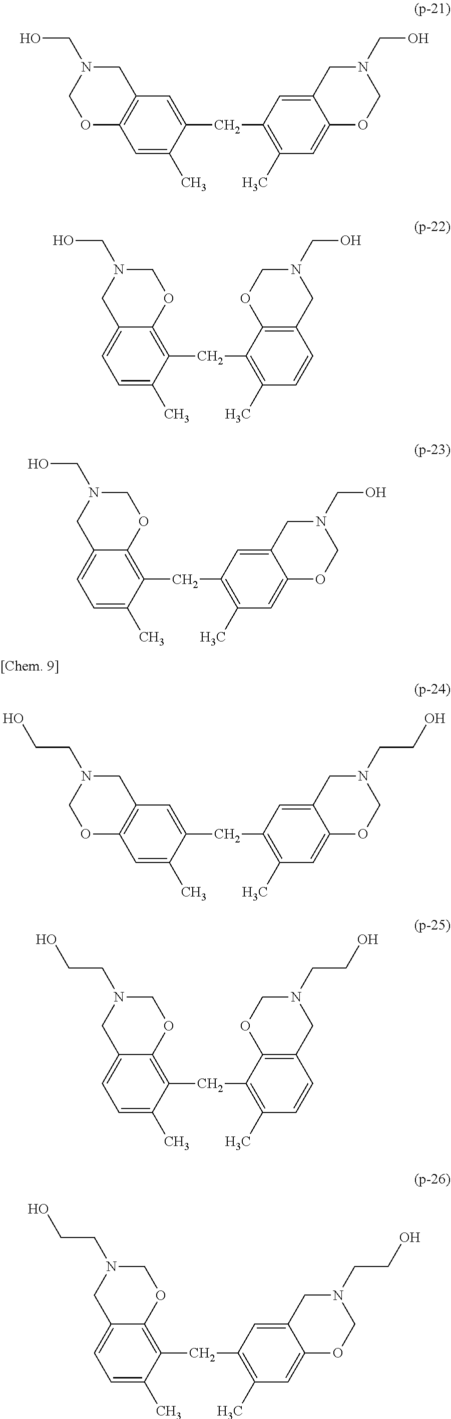 [SMILES: Cc1cc2c(cc1Cc1cc3c(cc1C)OCN(CO)C3)CN(CO)CO2 ; Cc1ccc2c(c1Cc1c(C)ccc3c1OCN(CO)C3)OCN(CO)C2 ; Cc1cc2c(cc1Cc1c(C)ccc3c1OCN(CO)C3)CN(CO)CO2 ; Cc1cc2c(cc1Cc1cc3c(cc1C)OCN(CCO)C3)CN(CCO)CO2 ; Cc1ccc2c(c1Cc1c(C)ccc3c1OCN(CCO)C3)OCN(CCO)C2 ; Cc1cc2c(cc1Cc1c(C)ccc3c1OCN(CCO)C3)CN(CCO)CO2]

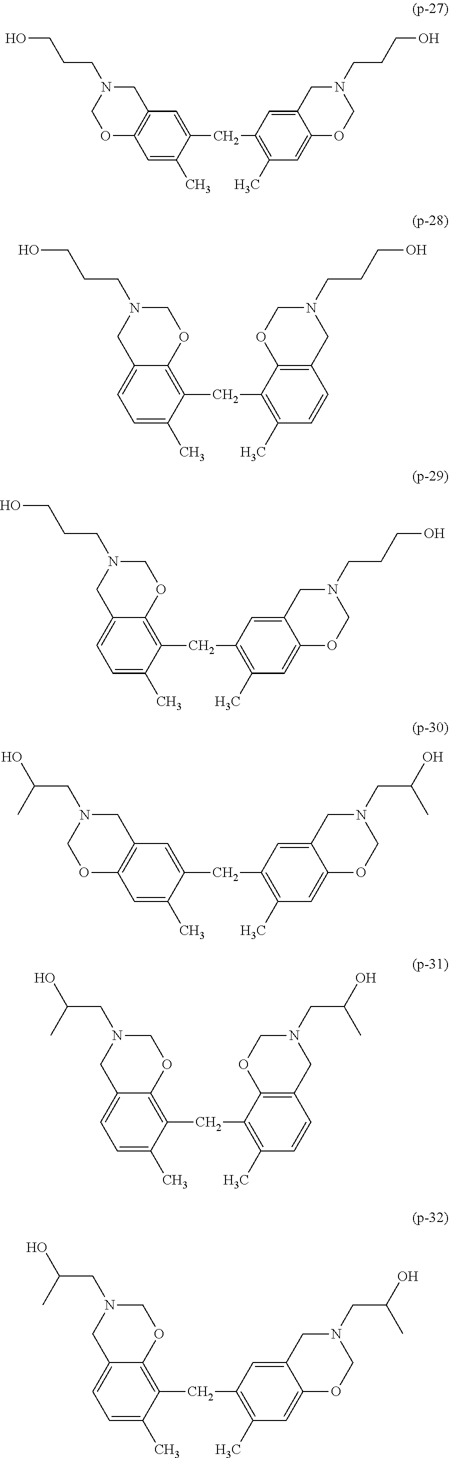 [SMILES: Cc1cc2c(cc1Cc1cc3c(cc1C)OCN(CCCO)C3)CN(CCCO)CO2 ; Cc1ccc2c(c1Cc1c(C)ccc3c1OCN(CCCO)C3)OCN(CCCO)C2 ; Cc1cc2c(cc1Cc1c(C)ccc3c1OCN(CCCO)C3)CN(CCCO)CO2 ; Cc1cc2c(cc1Cc1cc3c(cc1C)OCN(CC(C)O)C3)CN(CC(C)O)CO2 ; Cc1ccc2c(c1Cc1c(C)ccc3c1OCN(CC(C)O)C3)OCN(CC(C)O)C2 ; Cc1cc2c(cc1Cc1c(C)ccc3c1OCN(CC(C)O)C3)CN(CC(C)O)CO2]

### Method for Producing Inventive Compound

For the novel benzoxazine compound represented by general formula (1) in the present invention, there are no particular limitations on the starting materials in the production of the benzoxazine compound and the method for producing the benzoxazine compound. For example, as illustrated by the following reaction formula, a production method in which a bisphenol compound represented by general formula (2), an aminoalcohol compound represented by general formula (3), and formaldehyde are allowed to undergo dehydration condensation reaction to cyclize, thereby obtaining the target novel benzoxazine compound represented by general formula (1), may be used.

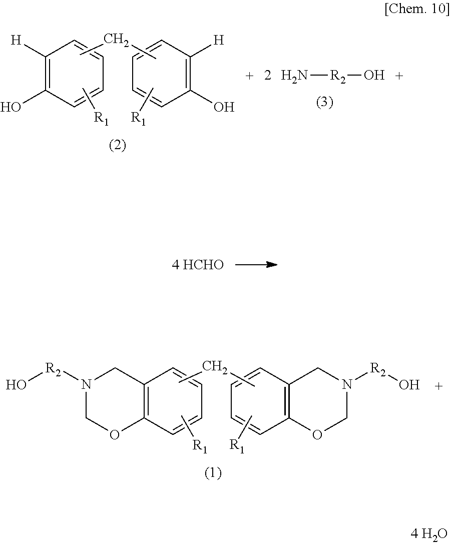

(In the formula, R₁ and R₂ are as defined in general formula (1).)

In the above production method, a bisphenol compound represented by general formula (2), an aminoalcohol compound represented by general formula (3), and a formaldehyde are used as starting materials.

Specific examples of the bisphenol compound represented by general formula (2) include bisphenol F (bis(2-hydroxyphenyl)methane, 2-hydroxyphenyl-4-hydroxyphenylmethane, bis(4-hydroxyphenyl)methane), bis(4-hydroxy-3-methylphenyl)methane, bis(2-hydroxy-5-methylphenyl)methane, bis(4-hydroxy-2-methylphenyl)methane, bis(2-hydroxy-6-methylphenyl)methane, and 2-hydroxy-6-methylphenyl-4-hydroxy-2-methyl-phenylmethane.

Specific examples of the aminoalcohol compound represented by general formula (3) include methanolamine, 2-aminoethanol, 3-amino-1-propanol, 1-amino-2-propanol, 4-amino-1-butanol, 2-amino-1-butanol, 4-amino-2-butanol, 5-amino-1-pentanol, 6-amino-1-hexanol, 7-amino-1-heptanol, and valinol. Of these, 2-aminoethanol is preferred.

Specific examples of the formaldehyde include an aqueous formaldehyde solution, 1,3,5-trioxane, and paraformaldehyde.

In the above production method, the amount of the formaldehyde used is preferably in the range of 4.0 to 20.0 mol, more preferably in the range of 4.0 to 16.0 mol, still more preferably in the range of 4.0 to 12.0 mol, relative to 1 mol of the bisphenol compound represented by general formula (2).

In the above production method, the amount of the aminoalcohol compound represented by general formula (3) used is preferably in the range of 2.0 to 10.0 mol, more preferably in the range of 2.0 to 8.0 mol, still more preferably in the range of 2.0 to 6.0 mol, relative to 1 mol of the bisphenol compound represented by general formula (2).

A catalyst for accelerating the reaction is not particularly necessary, but an acid catalyst or a base catalyst can be used as needed. In this case, examples of acid catalysts that can be used include, but are not limited to, concentrated hydrochloric acid, hydrochloric acid gas, trifluoroacetic acid, methanesulfonic acid, p-toluenesulfonic acid, benzoic acid, and mixtures thereof, and examples of base catalysts that can be used include, but are not limited to, sodium hydroxide, sodium carbonate, triethylamine, triethanolamine, and mixtures thereof.

The reaction is typically performed in the presence of a solvent. The solvent is not particularly limited as long as it does not inhibit the reaction, and preferred examples include toluene, xylene, ethyl acetate, butyl acetate, chloroform, dichloromethane, tetrahydrofuran, and dioxane. These solvents can be used alone or in combination. The amount of solvent used is not particularly limited as long as the reaction is not hindered, and is typically in the range of 0.5 to 5 times, preferably in the range of 1 to 3 times the amount of the bisphenol compound represented by general formula (2) on a weight basis.

The reaction temperature is typically in the range of 10° C. to 150° C., preferably in the range of 10° C. to 120° C., more preferably in the range of 10° C. to 80° C., still more preferably in the range of 20° C. to 70° C., particularly preferably in the range of 20° C. to 60° C.

The reaction may be performed under normal pressure conditions, or may be performed under increased pressure or reduced pressure.

In another embodiment, a process of removing water derived from the raw materials or water generated during the reaction out of the system may be included. The process of removing water generated from a reaction solution is not particularly limited and can be performed by distilling the generated water azeotropically with the solvent system in the reaction solution. The generated water can be removed out of the reaction system by using, for example, an isobaric dropping funnel equipped with a cock, a Dimroth condenser, or a Dean-Stark apparatus.

From the final reaction mixture obtained, the benzoxazine compound represented by general formula (1) can be obtained by a known method after completion of the reaction. For example, after the reaction, the remaining raw materials and solvent may be distilled off from the reaction mixture to thereby obtain the target as a residual liquid. Other possible methods include adding the residual liquid to a poor solvent to obtain the target as a precipitate, and adding a solvent to the reaction mixture to cause crystallization and performing filtration to obtain the target as powder or particles. The benzoxazine compound collected by any of these methods can be made into a high-purity product by, for example, standard purification means such as washing with a solvent or water or recrystallization.

### Resin Raw Material Composition Containing Benzoxazine Compound Represented by General Formula (1)

A resin raw material composition according to the present invention contains the benzoxazine compound represented by general formula (1) and can be obtained by distilling off the remaining raw materials and solvent from the reaction mixture described above. Alternatively, the target in the form of a precipitate can be obtained by adding the residual liquid to a poor solvent, or the resin raw material composition according to the present invention in the form of powder or particles can be obtained by adding a solvent to the reaction mixture to cause crystallization and performing filtration. For example, the resin raw material composition according to the present invention having a high content of the benzoxazine compound represented by general formula (1) can be obtained by performing standard purification such as washing with a solvent or water or recrystallization.

The resin raw material composition according to the present invention may be produced using, in the reaction for producing the benzoxazine compound represented by general formula (1), the bisphenol compound represented by general formula (2) composed of a mixture in which the position of the methylene chain bonded to the benzene rings varies.

The ratio of compounds in which the position of the methylene chain bonded to the benzene rings varies in the bisphenol compound represented by general formula (2) for use is not particularly limited.

Specifically, for example, when bisphenol F is used, a mixture of positional isomers thereof, that is, bis(2-hydroxyphenyl)methane, 2-hydroxyphenyl-4-hydroxyphenylmethane, and bis(4-hydroxyphenyl)methane, can be used, and the ratio thereof is not particularly limited.

Bisphenol F containing a large proportion of bis(2-hydroxyphenyl)methane can be obtained by, for example, the method of Japanese Unexamined Patent Application Publication No. 08-245464, and bisphenol F containing a large proportion of bis(4-hydroxyphenyl)methane can be obtained by, for example, the method of Japanese Unexamined Patent Application Publication No. 06-340565.

When such a mixture of positional isomers of bisphenol F and 2-aminoethanol as the aminoalcohol compound represented by general formula (3) are used to synthesize the benzoxazine compound represented by general formula (1) according to the present invention by the production method described above, a mixture of compounds (p-4), (p-5), and (p-6) can be obtained.

In the bisphenol compound represented by general formula (2) for use, the bisphenol (binuclear structure) content is not particularly limited but is preferably 50 wt % or more, more preferably 70 wt % or more, still more preferably 85 wt % or more, particularly preferably 89 wt % or more. A polynuclear structure, which is a by-product formed during the production of a bisphenol, may be contained.

The resin raw material composition in the present invention may contain a compound formed as a by-product in the reaction for producing the benzoxazine compound represented by general formula (1). The by-product may be, for example, a compound having a molecular weight higher than that of the benzoxazine compound represented by general formula (1).

In the resin raw material composition according to the present invention, the content of the benzoxazine compound represented by general formula (1) is not particularly limited. The content can be analyzed by gel permeation chromatography using a differential refractometer as a detector and is typically 10 to 100 area %, preferably 20 to 100 area %, more preferably 30 to 100 area %, particularly preferably 40 to 100 area %, relative to the area of all peaks detected in the analysis.

### Curable Resin Composition that Contains Benzoxazine Compound Represented by General Formula (1) or Resin Raw Material Composition Containing Benzoxazine Compound

The benzoxazine compound represented by general formula (1) or the resin raw material composition containing the benzoxazine compound according to the present invention can be used in the form of a curable resin composition containing the benzoxazine compound or the resin raw material composition as an essential component.

Examples of such forms include the benzoxazine compound represented by general formula (1) or the resin raw material composition containing the benzoxazine compound, and silicon oxide, aluminum oxide, magnesium oxide, boron nitride, aluminum nitride, silicon nitride, or silicon carbide, and include curable resin compositions obtained by mixing with an inorganic filler such as hexagonal boron nitride and a reinforcement fiber such as carbon fiber, glass fiber, organic fiber, boron fiber, steel fiber, or aramid fiber.

Examples of other forms include curable resin compositions that contain, as an essential component, the benzoxazine compound represented by general formula (1) or the resin raw material composition containing the benzoxazine compound and contain other polymeric materials.

The polymeric materials constituting the curable resin composition according to the present invention are not particularly limited, and raw materials of an epoxy resin, a phenol resin, a bismaleimide compound, and a benzoxazine compound other than the benzoxazine compound represented by general formula (1) can be contained.

Examples of the epoxy resin include ortho-cresol epoxy resins, biphenyl epoxy resins, biphenyl aralkyl epoxy resins, naphthalene epoxy resins, anthracene dihydride epoxy resins, and brominated novolac epoxy resins.

Examples of the phenol resin include novolac phenol resins such as phenol novolac resin, cresol novolac resin, naphthol novolac resin, aminotriazine novolac resin, and trisphenylmethane phenol novolac resin; modified phenol resins such as terpene-modified phenol resin and dicyclopentadiene-modified phenol resin; aralkyl resins such as phenol aralkyl resins having a phenylene backbone and/or a biphenylene backbone and naphthol aralkyl resins having a phenylene backbone and/or a biphenylene backbone; and resol phenol resins.

Examples of the bismaleimide compound include raw materials of the bismaleimide compound having the following structures.

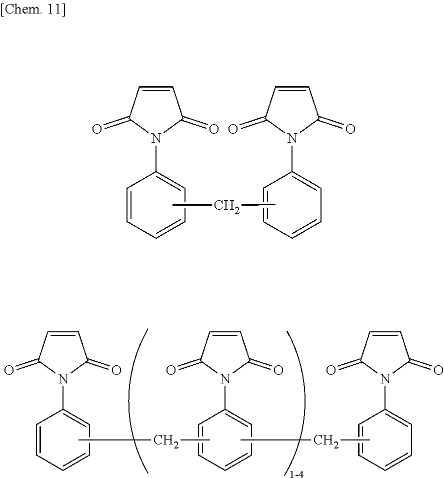

Examples of benzoxazine compounds other than the benzoxazine compound represented by general formula (1) include benzoxazine compounds having structures represented by general formulae (A) to (C) below.

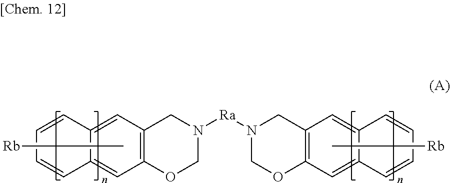

(In the formula, Ra represents a divalent group having 1 to 30 carbon atoms, each Rb independently represents an optionally substituted monovalent group having 1 to 10 carbon atoms, and n represents 0 or 1.)

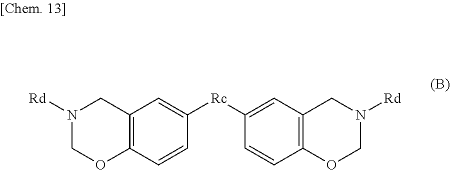

(In the formula, Rc represents a divalent group having 1 to 30 carbon atoms, a direct bond, an oxygen atom, a sulfur atom, a carbonyl group, or a sulfonyl group, and each Rd independently represents a monovalent group having 1 to 10 carbon atoms.)

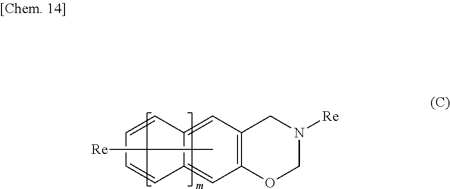

(In the formula, each Re independently represents a monovalent group having 1 to 10 carbon atoms, and m represents 0 or 1.)

Ra in the benzoxazine compound having the structure represented by general formula (A) represents a divalent group having 1 to 30 carbon atoms. Specific examples thereof include alkylene groups such as 1,2-ethylene, 1,4-butylene, and 1,6-hexylene; alkylene groups having a cyclic structure, such as 1,4-cyclohexylene, dicyclopentadienylene, and adamantylene; and arylene groups such as 1,4-phenylene, 4,4′-biphenylene, diphenyl ether-4,4′-diyl, diphenyl ether-3,4′-diyl, diphenyl ketone-4,4′-diyl, and diphenyl sulfone-4,4′-diyl.

Each Rb in the benzoxazine compound having the structure represented by general formula (A) independently represents a monovalent group having 1 to 10 carbon atoms. Specific examples thereof include alkyl groups such as a methyl group, an ethyl group, a propyl group, and a butyl group; alkenyl groups such as a vinyl group and an allyl group; alkynyl groups such as an ethynyl group and a propargyl group; and aryl groups such as a phenyl group and a naphthyl group, and these groups may further have a substituent such as an alkoxy group having 1 to 4 carbon atoms, an acyl group having 1 to 4 carbon atoms, a halogen atom, a carboxyl group, a sulfo group, an allyloxy group, a hydroxy group, or a thiol group.

Examples of the benzoxazine compound having the structure represented by general formula (A) include P-d type benzoxazine manufactured by Shikoku Chemicals Corporation, and JBZ-OP100N and JBZ-BP100N manufactured by JFE Chemical Corporation.

Rc in the benzoxazine compound having the structure represented by general formula (B) represents a divalent group having 1 to 30 carbon atoms, a direct bond, an oxygen atom, a sulfur atom, a carbonyl group, or a sulfonyl group. Examples of the divalent group having 1 to 30 carbon atoms include alkylene groups such as methylene, 1,2-ethylene, 1,4-butylene, and 1,6-hexylene; alkylene groups having a cyclic structure, such as 1,4-cyclohexylene, dicyclopentadienylene, and adamantylene; and alkylidene groups such as ethylidene, propylidene, isopropylidene, butylidene, phenylethylidene, cyclopentylidene, cyclohexylidene, cycloheptylidene, cyclododecylidene, 3,3,5-trimethylcyclohexylidene, and fluorenylidene.

Each Rd in the benzoxazine compound having the structure represented by general formula (B) independently represents a monovalent group having 1 to 10 carbon atoms. Specific examples thereof include alkyl groups such as a methyl group, an ethyl group, a propyl group, and a butyl group; alkenyl groups such as a vinyl group and an allyl group; alkynyl groups such as an ethynyl group and a propargyl group; and aryl groups such as a phenyl group and a naphthyl group, and these substituents may further have a substituent such as an alkoxy group having 1 to 4 carbon atoms, an acyl group having 1 to 4 carbon atoms, a halogen atom, a carboxyl group, a sulfo group, an allyloxy group, a hydroxy group (excluding the case where Rc is methylene), or a thiol group.

Examples of the benzoxazine compound having the structure represented by general formula (B) include F-a type benzoxazine manufactured by Shikoku Chemicals Corporation and BS-BXZ manufactured by Konishi Chemical Ind. Co., Ltd.

Each Re in the benzoxazine compound having the structure represented by general formula (C) independently represents a monovalent group having 1 to 10 carbon atoms. Specific examples thereof include alkyl groups such as a methyl group, an ethyl group, a propyl group, and a butyl group; alkenyl groups such as a vinyl group and an allyl group; alkynyl groups such as an ethynyl group and a propargyl group; and aryl groups such as a phenyl group and a naphthyl group, and these substituents may further have a substituent such as an alkoxy group having 1 to 4 carbon atoms, an acyl group having 1 to 4 carbon atoms, a halogen atom, a carboxyl group, a sulfo group, an allyloxy group, a hydroxy group, or a thiol group.

In particular, the curable resin composition according to the present invention preferably contains the benzoxazine compound represented by general formula (1) or the resin raw material composition containing the benzoxazine compound, and at least one selected from the group consisting of epoxy resins, benzoxazine compounds other than the benzoxazine compound represented by general formula (1), phenol resins, and bismaleimide compounds.

In the curable resin composition according to the present invention, the amount of the other polymeric materials mixed with the benzoxazine compound represented by general formula (1) or the resin raw material composition containing the benzoxazine compound is in the range of 0.01 parts by weight to 100 parts by weight relative to 1 part by weight of the benzoxazine compound represented by general formula (1) or the resin raw material composition containing the benzoxazine compound.

The curable resin composition according to the present invention can be obtained by adding the benzoxazine compound represented by general formula (1) or the resin raw material composition containing the benzoxazine compound to the other optional polymeric materials described above. The method of the addition is not particularly limited, and a method known in the art can be employed. Examples of the method include addition during synthesis or polymerization of the polymeric materials, addition of a resin formed of the polymeric materials to a molten resin melted in, for example, a melt extrusion step, and infiltration into, for example, a resin product formed of the polymeric materials.

The curable resin composition according to the present invention may entrain bubbles when cured if water or a residual solvent is contained in the composition, and thus to prevent this, it is preferable to perform a vacuum degassing treatment as a pretreatment. The vacuum degassing treatment may be performed at any temperature at which the curable resin composition according to the present invention is in a molten state but is preferably performed at up to 150° C. because curing does not proceed and degassing is facilitated. The vacuum degassing treatment is preferably, but not necessarily, performed at a low pressure (highly reduced pressure) and may be performed either in air or in a nitrogen-purged atmosphere. The vacuum degassing treatment is performed until no bubbles can be visually observed.

The curable resin composition according to the present invention may be silicon oxide, aluminum oxide, magnesium oxide, boron nitride, aluminum nitride, silicon nitride, or silicon carbide depending on the need in the application, and can be used as a mixture with an inorganic filler such as hexagonal boron nitride and a reinforcement fiber such as carbon fiber, glass fiber, organic fiber, boron fiber, steel fiber, or aramid fiber.

### Cured Product Obtained by Curing Curable Resin Composition According to Present Invention

Next, a cured product according to the present invention will be described.

The cured product according to the present invention can be obtained by curing the curable resin composition according to the present invention, which contains, as an essential component, the benzoxazine compound represented by general formula (1) or the resin raw material composition containing the benzoxazine compound according to the present invention.

Examples of the method for producing the cured product according to the present invention include curing by heating to a predetermined temperature; melting by heating, injecting into a mold or the like, and further heating the mold to achieve curing and molding; and injecting a melt into a preheated mold and curing the melt.

The cured product according to the present invention can be cured by performing ring-opening polymerization under the same curing conditions as those for ordinary benzoxazines. The curing temperature is typically in a temperature range of 150° C. to 300° C., preferably in a temperature range of 170° C. to 280° C., more preferably in a temperature range of 170° C. to 260° C., and is particularly preferably in a temperature range of 170° C. to 240° C. in order to provide a cured product with improved mechanical properties. When curing is performed in such a temperature range, the reaction time may be about 1 to 10 hours.

The production of the cured product may be performed either in air or in an inert gas atmosphere such as nitrogen, but is preferably performed in an inert gas atmosphere in order to prevent oxygen-induced degradation of the cured product obtained.

The resin composition according to the present invention can be cured by heat alone, but it is preferable to use a curing accelerator depending on, for example, the components other than the benzoxazine compound represented by general formula (1) and the content thereof. Examples of curing accelerators that can be used include, but are not limited to, tertiary amines such as 1,8-diaza-bicyclo[5.4.0]undecene-7, triethylenediamine, and tris(2,4,6-dimethylaminomethyl) phenol; imidazoles such as 2-ethyl-4-methylimidazole and 2-methylimidazole; phosphorus compounds such as triphenylphosphine, tetraphenylphosphonium bromide, tetraphenylphosphonium tetraphenylborate, and tetra-n-butylphosphonium-O,O-diethyl phosphorodithioate; quaternary ammonium salts; organic metal salts; and derivatives thereof. These may be used alone or in combination. Among these curing accelerators, tertiary amines, imidazoles, and phosphorus compounds are preferably used.

The benzoxazine compound represented by general formula (1) according to the present invention, because of having a lower curing temperature than that of Comparative Example Compound A above known in the art, enables higher efficiency due to reduced time of heating and cooling and saved energy in the process of molding a thermosetting resin and in addition can be used also for a heat-sensitive material (substrate), and thus is very useful. Furthermore, the cured product thereof has much higher heat resistance than Comparative Example Compound A above known in the art, and thus is a material excellent in stability and reliability at high temperatures and is very useful.

### EXAMPLES

The present invention will now be described more specifically with reference to Examples.

### Analysis Method

### 1. Reaction Solution Composition and Purity Analysis (Gel Permeation Chromatography: GPC)

The purity of benzoxazine compounds synthesized was defined as the area percentage of the benzoxazine compounds determined by this analysis.
Apparatus: HLC-8320/manufactured by Tosoh Corporation
Detector: differential refractometer (RI)

### Measurement Conditions

Flow rate: 1 mL/min
Eluate: tetrahydrofuran
Temperature: 40° C.
Wavelength: 254 nm
Measurement sample: One gram of a benzoxazine compound-containing composition was 200-fold diluted with tetrahydrofuran.

### 2. Curing Properties Evaluation

The curing properties evaluation of the benzoxazine compounds synthesized was performed by differential scanning calorimetry (DSC) under the following operating conditions. The exothermic peak temperature was used as a curing temperature.

### Measurement Conditions

Apparatus: DSC7020/manufactured by Hitachi High-Tech Science Corporation
Heating rate: 10° C./min
Measurement temperature range: 30° C. to 400° C.
Measurement atmosphere: nitrogen, 50 mL/min
Measurement sample: benzoxazine compounds synthesized, 3 mg

### 3. Heat Resistance Evaluation (5% Weight Loss Temperature Measurement)

The heat resistance evaluation of the benzoxazine compounds synthesized was performed based on their 5% weight loss temperature determined by thermogravimetry (hereinafter referred to as TG) that was performed under the following operating conditions after curing at 250° C. (curing time: 1 hour, heating rate: 10° C./min) and cooling to room temperature (cooling rate: 10° C./min).

### Measurement Conditions

Apparatus: DTG-60A/manufactured by Shimadzu Corporation
Temperature: 30° C. →500° C. (heating rate, 10° C./min)
Measurement atmosphere: open; nitrogen, 50 mL/min
Measurement sample: benzoxazine compounds synthesized, 10 mg

### 4. Heat Resistance Evaluation of Cured Product of Benzoxazine Compound

The heat resistance evaluation of cured products of the benzoxazine compounds synthesized was performed by glass transition temperature (Tg) measurement using dynamic viscoelasticity measurement under the following operating conditions.

### Measurement Conditions

Apparatus: DMA Q800 (manufactured by TA Instruments Japan Inc.)
Jig: dual cantilever
Frequency: 1 Hz
Temperature: 30° C.→250° C. (2° C./min)
Measurement sample: test pieces obtained by the method described later

### <Example 1> (Synthesis of Compound According to Present Invention Represented by Chemical Formula Below)

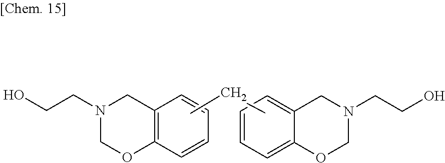

In a 1 L four-necked flask equipped with a thermometer, a stirrer, a condenser, and a dropping funnel, 97 g (0.49 mol) of bisphenol F (binuclear structure content, 90.1 wt %; isomer ratio thereof: bis(2-hydroxyphenyl)methane, 18.8 wt %; 2-hydroxyphenyl-4-hydroxyphenylmethane, 49.3 wt %; and bis(4-hydroxyphenyl)methane, 31.9 wt %; polynuclear structure content, 9.9 wt %), 62 g of 94% paraformaldehyde, and 121 g of toluene were loaded. After the reaction vessel was purged with nitrogen, the temperature of the mixed solution was adjusted to 70° C. Thereafter, 60 g of 2-aminoethanol was added dropwise into the four-necked flask using a dropping funnel over 2 hours while maintaining the temperature at 70° C. After completion of the dropwise addition, stirring was further performed at 70° C. for 3 hours. The composition of the reaction solution was analyzed by GPC according to the above analysis method, revealing that the percentage of the target compound present in the reaction solution was 51 area %.

After completion of the reaction, toluene and water were removed by reduced-pressure distillation at 70° C. The pressure during the distillation was gradually reduced so as to finally reach 4.8 kPa. The composition containing the target compound was taken out, solidified by cooling, then pulverized, and dried at 60° C. and 1.5 kPa to obtain 173 g of the target compound (purity: 53%, compounds with molecular weights higher than that of the target compound: 47 area %).

The results of ¹H-NMR analysis confirmed that a target compound having the above structure was obtained.

¹H-NMR analysis (400 MHz, solvent: CDCl₃, reference material: tetramethylsilane)

2.43-2.72 (2H, brm), 2.71-3.16 (4H, m), 3.41-4.09 (12H, m) , 4.69-5.01 (4H, m), 6.49-7.07 (6H, m).

### <Comparative Synthesis Example 1> (Synthesis of Comparative Example Compound A represented by Chemical Formula Below)

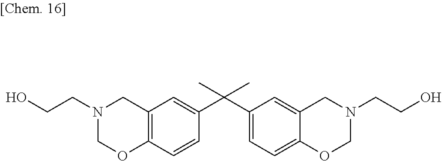 [SMILES: CC(C)(c1ccc2c(c1)CN(CCO)CO2)c1ccc2c(c1)CN(CCO)CO2]

In a 1 L four-necked flask equipped with a thermometer, a stirrer, a condenser, and a dropping funnel, 100 g (0.44 mol) of bisphenol A, 56 g of 94% paraformaldehyde, and 184 g of toluene were loaded. After the reaction vessel was purged with nitrogen, the temperature of the mixed solution was adjusted to 70° C. Thereafter, 53 g of 2-aminoethanol was added dropwise into the four-necked flask using a dropping funnel over 2 hours while maintaining the temperature at 70° C. After completion of the dropwise addition, stirring was further performed at 70° C. for 9.5 hours. The composition of the reaction solution was analyzed by GPC according to the above analysis method, revealing that the percentage of the target compound present in the reaction solution was 52 area %.

After completion of the reaction, toluene and water were removed by reduced-pressure distillation at 70° C. The pressure during the distillation was gradually reduced so as to finally reach 20 kPa. The composition containing Comparative Example Compound A was taken out to obtain 187 g of a Comparative Example Compound A-containing composition (purity: 54%, compounds with molecular weights higher than that of Comparative Example Compound A: 46 area %).

The results of ¹H-NMR analysis confirmed that a benzoxazine compound having the above chemical structure, i.e., Comparative Example Compound A, was obtained.

¹H-NMR analysis (400 MHz, solvent: CDCl₃, reference material: tetramethylsilane)

1.14-1.96 (6H, m), 2.45-2.77 (2H, brm), 2.78-3.18 (4H, m), 3.28-4.19 (10H, m), 4.70-5.14 (4H, m), 6.56-7.13 (6H, m).

### Comparative Synthesis Example 2

An Fa-type benzoxazine compound (Comparative Example Compound B) widely used as a benzoxazine compound and represented by the following structure was synthesized as described below.

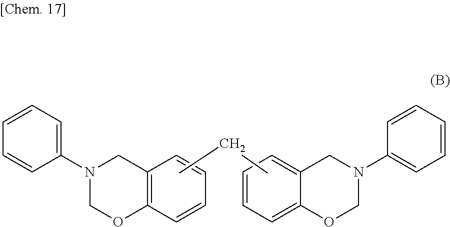

In a 1 L four-necked flask equipped with a thermometer, a stirrer, a condenser, and a dropping funnel, 83 g (0.41 mol) of bisphenol F, 77 g of aniline, 56 g of 94% paraformaldehyde, and 153 g of toluene were loaded. After the reaction vessel was purged with nitrogen, the temperature of the mixed solution was adjusted to 90° C. Thereafter, stirring was performed for 2 hours while maintaining the temperature at 90° C. The composition of the reaction solution was analyzed by GPC according to the above analysis method, revealing that the percentage of the target Comparative Example Compound B present in the reaction solution was 71 area %.

After completion of the reaction, toluene and water were removed by reduced-pressure distillation at 90° C. The pressure during the distillation was gradually reduced so as to finally reach 20 kPa. The composition containing Comparative Example Compound B was taken out to obtain 178 g of a Comparative Example Compound B-containing composition (purity: 69%, compounds with molecular weights higher than that of Comparative Example Compound B: 31 area %).

### Method of Preparing Test Piece of Cured Product of Example 1 Compound

A silicone casting plate for DMA measurement was filled with Comparative Example Compound A. Thereafter, heating at 175° C. for 2 hours in a dryer (DP32, manufactured by Yamato Scientific Co., Ltd.) followed by cooling was performed. The surface of the resulting plate-like resin cured product was polished with sandpaper to thereby prepare a test piece of the cured product.

### Curing Properties Evaluation and Heat Resistance Evaluation

For the benzoxazine compounds obtained in Example 1, Comparative Synthesis Example 1, and Comparative Synthesis Example 2 above, curing properties evaluation and heat resistance evaluation of cured products (5% weight loss temperature measurement and glass transition temperature (Tg) measurement) were performed according to the above analysis methods.

The results are listed in Table 1 below. In the table, “−” denotes unmeasured, and the glass transition temperature (Tg) of Comparative Example Compound B is a value given in Journal of the Japan Institute of Electronics Packaging, Vol. 14, No. 3, pp. 204 to 211, 2011.

TABLE 1

| | | Comparative | Comparative |
|---|---|---|---|
| | Example 1 | Example | Example |
| | Compound | Compound A | Compound B |
| Curing temperature | 224 | 243 | 250 |
| (° C.) | | | |
| 5% weight loss | 353 | 342 | 371 |
| temperature (° C.) | | | |
| Glass transition | 188 | — | 169 |
| temperature (Tg) | | | |

As shown in Table 1, it has become clear that Example 1 Compound, which is the inventive compound, cures at a lower temperature than Comparative Example Compound A and the widely used Fa-type benzoxazine compound (Comparative Example Compound B). This result indicates that the use of the novel benzoxazine compound represented by general formula (1) according to the present invention can lower the temperature during the process of molding a thermosetting resin, enabling higher efficiency due to reduced time of heating and cooling and saved energy, and in addition the benzoxazine compound can be used also for a heat-sensitive material (substrate) and thus is very useful.

As shown in Table 1, it has become clear that the cured product of Example 1 Compound, which is the inventive compound, is superior to the cured product of Comparative Example Compound A also in heat resistance when compared in terms of 5% weight loss temperature. It has become clear that as compared to the cured product of Comparative Example Compound B, the cured product of Example Compound 1 is somewhat inferior in 5% weight loss temperature but has heat resistance sufficient in applications in which the benzoxazine compound is used, and is superior in glass transition temperature (Tg).

1. A benzoxazine compound represented by general formula (1) below: 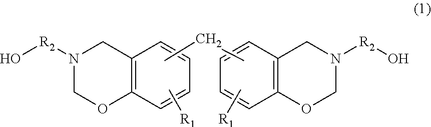
wherein R₁ represents a hydrogen atom or an alkyl group having 1 to 6 carbon atoms, and R₂ represents an alkylene group having 1 to 6 carbon atoms.

2. A resin raw material composition comprising the benzoxazine compound according to claim 1.

3. A curable resin composition comprising the benzoxazine compound according to claim 1.

4. The curable resin composition according to claim 3, further comprising at least one selected from the group consisting of epoxy resins, benzoxazine compounds other than the benzoxazine compound represented by general formula (1), phenol resins, and bismaleimide compounds.

5. A cured product obtained by curing the curable resin composition according to claim 3.

6. A curable resin composition comprising the resin raw material composition according to claim 2.

7. The curable resin composition according to claim 6, further comprising at least one selected from the group consisting of epoxy resins, benzoxazine compounds other than the benzoxazine compound represented by general formula (1), phenol resins, and bismaleimide compounds.

8. A cured product obtained by curing the curable resin composition according to claim 4.

9. A cured product obtained by curing the curable resin composition according to claim 6.

10. A cured product obtained by curing the curable resin composition according to claim 7.
